SEQUENCE LISTING (18 sequences)

SEQ ID NO: 1 - protein, 17 aa, Artificial Sequence
  Synthetic
GLAGGSAQSQRAPDRVL

SEQ ID NO: 2 - protein, 117 aa, Artificial Sequence
  Synthetic
QVQLQESGPGLVKPSETLSLTCTVSGYSITSDYAWNWIRQPPGKGLEWIGSISYSGITGYNPSLKSRVTISVDTSKNQFSLKLSSVTAADTAVYYCARMGYDGLAYWGQGTLVTVSS

SEQ ID NO: 3 - protein, 113 aa, Artificial Sequence
  Synthetic
DIVMTQTPLSLSVTPGQPASISCRSSQSIVHSNGNTYLEWYLQKPGQSPQLLIYKVSNRFSGVPDRFSGSGSGTEFTLKISRVEAEDVGVYYCFQGSHVPPTFGGGTKVEIKR

SEQ ID NO: 4 - protein, 447 aa, Artificial Sequence
  Synthetic
QVQLQESGPGLVKPSETLSLTCTVSGYSITSDYAWNWIRQPPGKGLEWIGSISYSGITGYNPSLKSRVTISRDTSKNQFSLKLSSVTAADTAVYYCARMGYDGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 219 aa, Artificial Sequence
  Synthetic
DIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 52 aa, Artificial Sequence
  Synthetic
GLAGGSAQSQRAPDRVLCHSGQQQGLPRAAGGSVPHPRCHCGAGRADWPGPP

SEQ ID NO: 7 - protein, 12 aa, Artificial Sequence
  Synthetic
GLAGGSAQSQRA

SEQ ID NO: 8 - protein, 117 aa, Artificial Sequence
  Synthetic
QVQLQESGPGLVKPSETLSLTCTVSGYSITSDYAWNWIRQPPGKGLEWMISISYSGITGYNPSLKSRVTISVDTSKNQFSLKLSSVTAADTAVYYCARMGYDGLAYWGQGTLVTVSS

SEQ ID NO: 9 - protein, 117 aa, Artificial Sequence
  Synthetic
QVQLQESGPGLVKPSETLSLTCTVSGYSITSDYAWNWIRQPPGKGLEWIGSISYSGITGYNPSLKSRVTISRDTSKNQFSLKLSSVTAADTAVYYCARMGYDGLAYWGQGTLVTVSS

SEQ ID NO: 10 - protein, 113 aa, Artificial Sequence
  Synthetic
DIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPPTFGGGTKVEIKR

SEQ ID NO: 11 - protein, 466 aa, Artificial Sequence
  Synthetic
MEFGLSWLFLVAILKGVQCQVQLQESGPGLVKPSETLSLTCTVSGYSITSDYAWNWIRQPPGKGLEWIGSISYSGITGYNPSLKSRVTISRDTSKNQFSLKLSSVTAADTAVYYCARMGYDGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 239 aa, Artificial Sequence
  Synthetic
MRVPAQLLGLLLLWLPGARCDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 11 aa, Artificial Sequence
  Synthetic
GYSITSDYAWN

SEQ ID NO: 14 - protein, 16 aa, Artificial Sequence
  Synthetic
SISYSGITGYNPSLKS

SEQ ID NO: 15 - protein, 9 aa, Artificial Sequence
  Synthetic
RMGYDGLAY

SEQ ID NO: 16 - protein, 16 aa, Artificial Sequence
  Synthetic
RSSQSIVHSNGNTYLE

SEQ ID NO: 17 - protein, 7 aa, Artificial Sequence
  Synthetic
KVSNRFS

SEQ ID NO: 18 - protein, 9 aa, Artificial Sequence
  Synthetic
FQGSHVPPT

(19) United States
(12) Patent Application Publication
(10) Pub. No.: US 2024/0191000 A1
(43) Pub. Date: Jun. 13, 2024

(54) HIGH CONCENTRATION ANTIBODY FORMULATIONS

(71) Applicant: ONENESS BIOTECH CO., LTD., Taipei City (TW)

(72) Inventors: Nien-Yi CHEN, Taipei City (TW); Wei-Ting KAO, Taipei City (TW)

(73) Assignee: ONENESS BIOTECH CO., LTD., Taipei City (TW)

(21) Appl. No.: 18/553,228

(22) PCT Filed: Apr. 1, 2022
(86) PCT No.: PCT/CN2022/084711

§ 371 Date: Sep. 29, 2023

Related U.S. Application Data
(60) Provisional application No. 63/170,042, filed on Apr. 2, 2021.

(51) Int. Cl. C07K 16/42; A61K 39/00; A61K 47/02; A61K 47/18; A61K 47/26

(52) U.S. Cl. CPC C07K 16/4291; A61K 47/02; A61K 47/183; A61K 47/26; A61K 2039/505; A61K 2039/545; C07K 2317/24; C07K 2317/565

(57) ABSTRACT

A high concentration antibody formulation comprising an antibody at a concentration of about 120-200 mg/ml, a histidine buffer at a concentration of about 5-15 mM, an amino acid at a concentration of about 1-1.5% (w/v), a tonicity modifier such as sodium chloride at a concentration of about 50-100 mM; and a non-ionic surfactant such as polysorbate 80 at a concentration of about 0.005-0.02% (w/v). In some instances, the high concentration antibody formulation may have a pH of about 5.5 to 6.5.

### CROSS REFERENCE TO RELATED APPLICATIONS

This application claims the benefit of the filing date of U.S. Provisional Application Ser. No. 63/170,042, filed Apr. 2, 2021, the entire contents of which are incorporated by reference herein.

### BACKGROUND OF THE INVENTION

Formulations with high concentration of biologic molecules such as recombinant proteins and monoclonal antibodies are usually required for certain drug delivery routes, for example, subcutaneous injection. While high concentration antibody formulations have been developed for certain antibody drugs, it remains challenging to produce highly concentrated antibody formulations (e.g., >100 mg/ml), including irreversible aggregation, irreversible precipitation, and/or high viscosity.

### SUMMARY OF THE INVENTION

The present disclosure is based, at least in part, on the development of high concentration antibody formulations, which show superior stability under various conditions and over as along as a two-year storage period and superior injectability.

Accordingly, one aspect of the present disclosure features an antibody formulation, comprising: (a) an antibody binding to a CmX domain of a membrane-bound IgE at a concentration of about 120-200 mg/ml, (b) histidine at a concentration of about 5-15 mM, (c) an amino acid at a concentration of about 1-3.0% (w/v), wherein the amino acid is arginine or threonine; (d) sodium chloride at a concentration of about 50-100 mM; and (e) polysorbate 80 at a concentration of about 0.005-0.02%. Such an antibody formulation may have a pH of about 5.5 to 6.5. In some embodiments, the antibody formulation consists essentially the components of (a)-(e). Alternatively or in addition, antibody formulation is free of a sugar- or sugar alcohol-based stabilizer. Examples include sucrose, sorbitol, or trehalose.

In some embodiments, the antibody formulation may comprise about 150 mg/ml of the antibody, about 10 mM of the histidine, about 1.25% of the amino acid, about 75 mM of the sodium chloride, and about 0.01% of the polysorbate 80. In some examples, the amino acid in the antibody formulation may be arginine and the antibody formulation has a pH of about 6.0 to 6.5. In other examples, the amino acid in the antibody formulation is threonine and the antibody formulation has a pH of about 6.0.

In any of the antibody formulations disclosed herein, the antibody contained therein that binds the Cεmx domain of a membrane-bound IgE may comprise the same heavy chain complementary determining regions (CDRs) as antibody FB825; and/or the same light chain complementary determining regions (CDRs) as antibody FB825. In some embodiments, the antibody is a human antibody or a humanized antibody. In some embodiments, the antibody is a full-length antibody.

In some embodiments, the antibody comprises a heavy chain variable region (VH) having the amino acid sequence of SEQ ID NO:2, SEQ ID NO:8, or SEQ ID NO:9, and a light chain variable region (VL) having the amino acid sequence of SEQ ID NO:3 or SEQ ID NO: 10. In some examples, the antibody comprises a VH of SEQ ID NO:9 and a VL of SEQ ID NO:10. In some examples, the antibody is an IgG1 molecule. In specific examples, the antibody comprises a heavy chain comprising the amino acid sequence of SEQ ID NO: 4 or SEQ ID NO: 11 and a light chain comprising the amino acid sequence of SEQ ID NO:5 or SEQ ID NO:12.

In other aspects, provided herein is a pre-filled syringe, comprising any of the high concentration antibody formulations disclosed herein. In some embodiments, the volume of the antibody formulation in the pre-filled syringe is about 1-2 ml.

In yet other aspects, the present disclosure features a method for treating a disorder associated with immunoglobulin E (IgE), the method comprising administering to a subject in need thereof an effective amount of any of the antibody formulations disclosed herein. In some embodiments, the subject receives one dose of the antibody formulation. In other embodiments, the subject receives at least two doses of the antibody formulation. In some instances, two consecutive doses are administered to the subject at least 3 months apart. In any method disclosed herein, the antibody formulation is administered subcutaneously.

In some embodiments, the subject is a human patient having or suspected of having allergic asthma, allergic rhinitis, atopic dermatitis, or hyper IgE syndrome. In some examples, the human patient has atopic dermatitis.

Also within the scope of the present disclosure are any of the antibody formulations disclosed herein for use in treating a disorder associated with immunoglobulin E (IgE) such as those disclosed herein, and use of the antibody formulation for manufacturing a medicament for use in treating the target disorder.

The details of one or more embodiments of the invention are set forth in the description below. Other features or advantages of the present invention will be apparent from the following drawings and detailed description of several embodiments, and also from the appended claims.

### DETAILED DESCRIPTION OF THE INVENTION

In atopic individuals who are at increased risk of developing allergies, the IgE concentration in the circulatory system could be elevated, for example, to a level at least 10 times higher than the normal level. The concentration of allergen-specific IgE antibody is closely correlated with clinical symptoms and may be over 1000 times higher in patients with allergic diseases than in healthy individuals. Immunoglobulin E sensitizes effector cells such as basophils, mast cells, and activated eosinophils by occupying the high-affinity IgE receptor, FcεRI, on which they are expressed. In type I hypersensitivity, allergens cross-link IgE molecules bound by FcεRI and subsequently trigger the degranulation of effector cells, releasing proinflammatory mediators, such as histamines and leukotrienes. The IgE-mediated allergic pathway, which generates mediator-related allergic symptoms, initiates immune activities locally or systemically. Basophils and mast cells also release a wide spectrum of inflammatory cytokines and chemokines that not only cause clinical symptoms directly but also activate and recruit various cell types to augment inflammatory status. Hence, anti-IgE therapy can attenuate both the IgE-mediated pathway and inflammatory conditions.

Multiple anti-IgE antibodies have been developed for treatment of IgE-associated allergic disorders. However, it still remains challenging to develop high concentration antibody formulations for delivery of such antibodies via a non-intravenous infusion route, for example, via subcutaneous injection.

The present disclosure is based, at least in part, on the development of a high concentration antibody formulation comprising an exemplary anti-IgE antibody (FB825), which showed superior stability under various conditions and over as along as a two-year storage period and superior injectability. Accordingly, provided herein are high concentration antibody formulations and uses thereof for treating IgE-associated allergic disorders.

### I. High Concentration Antibody Formulation

In some aspects, provided herein are high concentration antibody formulations comprising any of the anti-IgE antibodies disclosed herein. As used herein, “a high concentration formulation” refers to a formulation comprising a biologic molecule such as an antibody at a concentration of at least 100 mg/ml. The high concentration antibody formulations may comprise, in addition to the anti-IgE antibody, a suitable buffering agent, a suitable amino acid excipient, a suitable tonicity modifier, and a suitable non-ionic surfactant. Such a high concentration antibody formulation may have a pH of about 5.5-6.5.

The high concentration antibody formulations disclosed herein may possess one or more of the following superior features: (a) stable under conditions such as agitation (e.g., for 4 hours at room temperature), freeze/thaw (e.g., at least 5 consecutive cycles), UV light exposure, (b) stable upon short-term storage (e.g., at 40° ° C. for 8 weeks, 45° C. for two weeks, and/or 55° C. for one week) and/or long-term storage (e.g., for two-years under various temperature conditions), (c) suitable physical features such as turbidity (e.g., A650<0.01 such as around 0.006-0.008), osmolality (e.g., isotonic; around 300 mOsm)), viscosity, and/or injectability (e.g., require less than 2 lb_{f} to expel 1 ml of the formulation in 5-10 seconds or less than 5 seconds to deliver 1 ml of the formulation using 3-5 lb_{f}). In some instances, stability can be indicated by the percentage change of the main peak, low molecular weight peak, and/or high molecular weight peak as analyzed by SEC-HPLC and main peak, acidic peak, basic peak as analyzed by SCX-HPLC following routine practice and/or disclosures provided in Examples below.

A. Antibodies Capable of Binding to a CεmX Domain of a Membrane-Bound IgE In some instances, the anti-IgE antibody in the high concentration antibody formulations disclosed herein can be an antibody that binds the CεmX domain of a membrane-bound IgE molecule. CmX is a 52-amino acid segment located between the CH4 domain and the C-terminal membrane-anchoring segment of human membrane-bound ε chain (me). The amino acid sequence of an exemplary CεmX fragment of human mIgE is provided below (SEQ ID NO:6):
GLAGGSAQSQ RAPDRVLCHS GQQQGLPRAA GGSVPHPRCH CGAGRADWPG PP

The antibodies described herein can bind to the CεmX domain of a mIgE, for example, mIgE expressed on the surface of B cells. Such antibodies may induce cell death of the B cells expressing mIgE via, for example, antibody-dependent cell cytotoxicity and/or cell apoptosis, thereby eliminate the B cells, which would lead to reduced production of free IgE. Accordingly, the anti-CεmX antibodies described herein can reduce the level of total IgE in a subject (e.g., a human patient) being treated with the antibody.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used in the methods described herein can be murine, rat, human, or any other origin (including chimeric or humanized antibodies).

Any of the antibodies described herein can be either monoclonal or polyclonal. A “monoclonal antibody” refers to a homogenous antibody population and a “polyclonal antibody” refers to a heterogenous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some examples, the antibody disclosed herein specifically binds a CεmX domain of a membrane-bound IgE, which may be expressed on the surface of a B cell. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a CεmX domain epitope is an antibody that binds this CεmX domain epitope with greater affinity, avidity, more readily, and/or with greater duration than it binds to other CεmX domain epitopes or non-CεmX domain epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

The binding affinity of an anti-CεmX antibody described herein can be less than about 100 nM, e.g., less than about 50 nM, about 10 nM, about 1 nM, about 500 pM, about 100 pM, or about 50 pM to any of about 2 pM. Binding affinity can be expressed K_{D} or dissociation constant, and an increased binding affinity corresponds to a decreased K_{D}. One way of determining binding affinity of antibodies to CεmX is by measuring binding affinity of monofunctional Fab fragments of the antibody. To obtain monofunctional Fab fragments, an antibody (for example, IgG) can be cleaved with papain or expressed recombinantly. The affinity of an anti-CεmX Fab fragment of an antibody can be determined by surface plasmon resonance (BIAcore3000™ surface plasmon resonance (SPR) system, BIAcore, INC, Piscaway N.J.). Kinetic association rates (kₒₙ) and dissociation rates (k_{off}) (generally measured at 25° ° C.) are obtained; and equilibrium dissociation constant (K_{D}) values are calculated as k_{off}/kₒₙ.

In some embodiments, the antibody binds the CεmX domain of a human IgE, and does not significantly bind an IgE from another mammalian species. In some embodiments, the antibody binds human IgE as well as one or more IgE from another mammalian species. The epitope(s) bound by the antibody can be continuous or discontinuous.

In some embodiments, the anti-CεmX antibody described herein binds an N-terminal portion of the CεmX domain, e.g., GLAGGSAQSQRAPDRVL (SEQ ID NO:1) or GLAGGSAQSQRA (SEQ ID NO:7). Such an antibody may have the same heavy chain and/or light chain CDRs as antibody FB825. See also U.S. Pat. No. 8,460,664, the relevant disclosures therein are incorporated by reference herein. The anti-CεmX antibody may be a humanized antibody. In some examples, the anti-CεmX antibody for use in the methods described herein is FB825, or a functional variant thereof. See also U.S. Pat. No. 8,460,664, and US20200297815, the relevant disclosures therein are incorporated by reference herein.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/)

A functional variant (equivalent) of FB825 has essentially the same epitope-binding specificity as FB825 and exhibits substantially similar bioactivity as FB825, including the activity of eliminating B cells expressing mIgE and reducing the level of total IgE in a subject. In some embodiments, a functional variant of FB825 contains the same regions/residues responsible for antigen-binding as FB825, such as the same specificity-determining residues in the CDRs or the whole CDRs. In other embodiments, a functional variant of FB825 comprises a V_{H} chain that includes a V_{H} CDR1, V_{H} CDR2, and V_{H} CDR3 at least 75% (e.g., 80%, 85%, 90%, 95%, or 98%) identical to the corresponding V_{H} CDRs of FB825, and a V_{L} chain that includes a V_{L} CDR1, V_{L} CDR2, and V_{L} CDR3 at least 75% (e.g., 80%, 85%, 90%, 95%, or 98%) identical to the corresponding V_{H} CDRs of FB825. For example, a functional variant of FB825 may comprise a V_{H} chain that includes up to 5 (e.g., 1, 2, 3, 4, or 5) amino acid residue variations in the V_{H} CDR regions (V_{H} CDR1, CDR2, and/or CDR3 in total) as compared to the V_{H} CDRs of FB825, and/or a V_{L} chain that includes up to 5 (e.g., 1, 2, 3, 4, or 5) amino acid residue variations in the V_{L} CDR regions (V_{L} CDR1, CDR2, and/or CDR3 in total) as compared to the V_{H} CDRs of FB825.

Alternatively, the functional variant of FB825 comprises a V_{H} chain at least 75% (e.g., 80%, 85%, 90%, 95%, or 98%) identical to the V_{H} chain of FB825 and a V_{L} chain at least 75% (e.g., 80%, 85%, 90%, 95%, or 98%) identical to the V_{L} chain of FB825. The amino acid sequence variations may occur only in one or more of the V_{H} and/or V_{L} framework regions.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-CεmX antibody for use in the treatment method disclosed herein may have one of the following heavy chain variable regions (CDRs following the Kabat definition are in boldface and underlined. SEQ ID NOs: 13-15 for heavy chains CDRs 1-3, respectively):

(SEQ ID NO: 2)
QVQLQESGPGLVKPSETLSLTCTVSGYSITSDYAWNWIRQPPGKGLEWI
GSISYSGITGYNPSLKSRVTISVDTSKNQFSLKLSSVTAADTAVYYCAR
MGYDGLAYWGQGTLVTVSS
(SEQ ID NO: 8)
QVQLQESGPGLVKPSETLSLTCTVSGYSITSDYAWNWIRQPPGKGLEWM
ISISYSGITGYNPSLKSRVTISVDTSKNQFSLKLSSVTAADTAVYYCAR
MGYDGLAYWGQGTLVTVSS
(SEQ ID NO: 9)
QVQLQESGPGLVKPSETLSLTCTVSGYSITSDYAWNWIRQPPGKGLEWI
GSISYSGITGYNPSLKSRVTISRDTSKNQFSLKLSSVTAADTAVYYCAR
MGYDGLAYWGQGTLVTVSS

Alternatively or in addition, the anti-CεmX antibody for use in the treatment method disclosed herein may have one of the following light chain variable regions (CDRs following the Kabat definition are in boldface and underlined. SEQ ID NOs: 16-18 for light chain CDRs 1-3, respectively):

(SEQ ID NO: 3)
DIVMTQTPLSLSVTPGQPASISCRSSQSIVHSNGNTYLEWYLQKPGQSP
QLLIYKVSNRFSGVPDRFSGSGSGTEFTLKISRVEAEDVGVYYCFQGSH
VPPTFGGGTKVEIKR
(SEQ ID NO: 10)
DIVMTQTPLSLSVTPGQPASISCRSSQSIVHSNGNTYLEWYLQKPGQSP
QLLIYKVSNRFSGVPDRFSGSGSGTDFTLKISRVEAEDVGVYYCFQGSH
VPPTFGGGTKVEIKR

In some embodiments, the heavy chain of any of the anti-IgE antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. In some examples, the heavy chain constant region is of human origin. Alternatively or in addition, the light chain of the anti-IgE antibody may further comprise a light chain constant region (CL), which can be any CL known in the art, e.g., a human light chain constant region. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some instances, the anti-IgE antibody is FB825, which is an IgG1 molecule having a heavy chain and a light chain provided below, which comprise the V_{H} of SEQ ID NO:2 and the V_{L} of SEQ ID NO:3, respectively. The heavy chain (top) and light chain (bottom) amino acid sequence of FB825 in full-length format is provided below (including N-terminal signal peptide sequences, which are italicized).

(SEQ ID NO: 11)
MEFGLSWLFLVAILKGVQCQVQLQESGPGLVKPSETLSLTCTVSGYSITSDYAWNWIRQPPGKGLEWIGS
ISYSGITGYNPSLKSRVTISRDTSKNQFSLKLSSVTAADTAVYYCARMGYDGLAYWGQGTLVTVSSASTK
GPSVFPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
(SEQ ID NO: 12)
MRVPAQLLGLLLLWLPGARCDIVMTQTPLSLSVTPGQPASISCRSSQSIVHSNGNTYLEWYLQKPGQSPQ
LLIYKVSNRFSGVPDRFSGSGSGTDFTLKISRVEAEDVGVYYCFQGSHVPPTFGGGTKVEIKRTVAAPSV
FIFPPSDEQLKSGTASVVCLLNNFYPREAKVQWKVDNALQSGNSQESVTEQDSKDSTYSLSSTLTLSKAD
YEKHKVYACEVTHQGLSSPVTKSFNRGEC

SEQ ID NO:4 represents the amino acid sequence of the mature heavy chain of FB825 (with no N-terminal signal peptide sequence) and SEQ ID NO:5 represents the amino acid sequence of the mature light chain of FB825 (with no N-terminal signal peptide sequence).

In some examples, the FB825 antibody may comprise a heavy chain comprising the amino acid sequence of SEQ ID NO:4 and a light chain comprising the amino acid sequence of SEQ ID NO:5. Alternatively, the heavy chain and/or the light chain of FB825 may contain an N-terminal signal peptide, e.g., those described above. In some instances, the FB825 antibody may comprise a heavy chain comprising the amino acid sequence of SEQ ID NO:11 and a light chain comprising the amino acid sequence of SEQ ID NO: 12.

### B. Antibody Preparation

Antibodies capable of binding a CεmX domain of a membrane-bound IgE as described herein can be made by any method known in the art. See, for example, Harlow and Lane, (1988) Antibodies: A Laboratory Manual, Cold Spring Harbor Laboratory, New York.

In some embodiments, antibodies specific to a target antigen (e.g., a CεmX domain of a mIgE such as a human mIgE) can be made by the conventional hybridoma technology. If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in reducing total IgE. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XenomouseR™ from Amgen, Inc. (Fremont, Calif.) and HuMAb-MouseR™ and TC Mouse™ from Medarex, Inc. (Princeton, N.J.). In another alternative, antibodies may be made recombinantly by phage display technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)₂ fragments can be produced by pepsin digestion of an antibody molecule, and Fab fragments that can be generated by reducing the disulfide bridges of F(ab′)₂ fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

Alternatively, antibodies capable of binding to the target antigens as described herein (an IgE molecule or a fragment thereof) may be isolated from a suitable antibody library via routine practice. Antibody libraries can be used to identify proteins that bind to a target antigen (e.g., human IgE such as the CεmX fragment thereof) via routine screening processes. In the selection process, the polypeptide component is probed with the target antigen or a fragment thereof and, if the polypeptide component binds to the target, the antibody library member is identified, typically by retention on a support. Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

There are a number of routine methods known in the art to identify and isolate antibodies capable of binding to the target antigens described herein, including phage display, yeast display, ribosomal display, or mammalian display technology.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the IgE polypeptide have been replaced (swapped) with sequences from a closely related, but antigenically distinct protein (such as another member of the immunoglobulin protein family). By assessing binding of the antibody to the mutant immunoglobulin, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-IgE antibody disclosed herein such as FB825 can be prepared by recombinant technology as exemplified below.

Nucleic acids encoding the heavy and light chain of an anti-CεmX antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16): 1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColEl for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-CεmX antibody, as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-IgE antibody and the other encoding the light chain of the anti-IgE antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-CεmX antibody as described herein, vectors (e.g., expression vectors) containing such; and host cells comprising the vectors are within the scope of the present disclosure.

### C. High Concentration Antibody Formulations

Any of the anti-IgE antibodies disclosed herein, e.g., FB825, may be used for preparing the high concentration antibody formulations. In addition to the anti-IgE antibody, the high concentration antibody formulation disclosed herein may further comprise a suitable buffering agent (e.g., histidine at a concentration of about 5-15 mM), an amino acid excipient (e.g., arginine or threonine at a concentration of about 1-3%, w/v), a tonicity modifier (e.g., sodium chloride at a concentration of about 50-100 mM), and a non-ionic surfactant (e.g., polysorbate 80 at a concentration of about 0.005-0.02%). The antibody formulation may have a pH of about 5.5 to 6.5. In some examples, the antibody formulation may have a pH value of about 6.0 to about 6.5. In specific examples, the antibody formulation has a pH value of about 6.0.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The anti-IgE antibody in the high concentration formulation may be at a concentration of at least 100 mg/ml. In some embodiments, the high concentration formulation contains the antibody at a concentration of about 100 mg/ml to about 250 mg/ml, for example, about 100 mg/ml to about 150 mg/ml, about 125 mg/ml to about 150 mg/ml, about 150 mg/ml to about 180 mg/ml, about 150 mg/ml to about 200 mg/ml, about 180 mg/ml to about 200 mg/ml or about 200 mg/ml to about 250 mg/ml. In some examples, the high concentration antibody formulation may comprise the antibody (e.g., FB825) at a concentration of about 130-165 mg/ml, for example, about 150 mg/ml.

The high concentration antibody formulation disclosed herein further comprises a buffering agent. A buffering agent refers to a weak acid or a weak base that helps maintain the pH of an aqueous solution. In some instances, the buffering agent used in the high concentration antibody formulation is histidine, which may be at a concentration of about 5-15 mM. In some examples, the histidine is at a concentration of about 5-8 mM, 5-10 mM, 10-15 mM, or 12-15 mM. In specific examples, the histidine is at a concentration of about 10 mM.

Further, the high concentration antibody formulation may comprise an amino acid excipient, which may be at a concentration of about 1-3% (w/v). In some embodiments, the amino acid excipient is arginine. In some examples, the arginine is at a concentration of about 1-1.3% (w/v), for example, about 1.25% (w/v). In other examples, the arginine may be at a concentration of about 2-3% (w/v), for example, about 2.5% (w/v). In some embodiments, the amino acid excipient is threonine. In some examples, the threonine is at a concentration of about 1-1.3% (w/v), for example, about 1.25% (w/v). In other examples, the threonine may be at a concentration of about 2-3% (w/v), for example, about 2.5% (w/v).

In addition, the high concentration antibody formulation disclosed herein further comprises a tonicity modifier. With respect to solution, tonicity is a property in reference to a particular membrane (e.g., cell membrane) and is equal to the sum of the concentration of the solutes in the solution (e.g., an aqueous formulation), which have the capacity to exert an osmotic force across the membrane. A tonicity modifier adjusts the tonicity of the formulation. In some embodiments, the tonicity modifier contained in the high concentration antibody formulation disclosed herein is sodium chloride, which may be at a concentration of about 50-100 mM. In some examples, the concentration of the tonicity modifier (e.g., sodium chloride) is about 50-80 mM, about 60-80 mM, or about 70-80 mM. In specific examples, the tonicity modifier is sodium chloride at a concentration of about 75 mM.

Moreover, the high concentration antibody formulation disclosed herein further comprises a non-ionic surfactant. A non-ionic surfactant is a type of surfactant that does not carry a charge on its hydrophilic head group and therefore has no net electrical charge in their formulations. In some embodiments the non-ionic surfactant in the high concentration antibody formulation disclosed herein is polysorbate 80, which may be at a concentration of about 0.005-0.02% (w/v). In some examples, the concentration of the non-ionic surfactant such as polysorbate 80 may range from 0.008-0.015%. In specific examples, the non-ionic surfactant is polysorbate 80 at a concentration of about 0.01%.

In some examples, the high concentration antibody formulation disclosed herein may comprise about 150 mg/ml of an anti-IgE antibody (e.g., FB825), about 10 mM of the histidine, about 1.25% of the amino acid, about 75 mM of the sodium chloride, and about 0.01% of the polysorbate 80.

In some examples, the high concentration antibody formulation disclosed herein consists essentially of the anti-IgE antibody, the buffering agent, the amino acid excipient, the tonicity modifier, and the non-ionic surfactant. Such a formulation does not contain components that would materially affect the basic and novel characteristic of the formulation.

In some example, the high concentration antibody formulation is substantially free of (e.g., completely free of) a sugar-based or a sugar alcohol-based stabilizer. For example, the formulation is substantially free of (e.g., completely free of) sucrose. Alternatively, the formulation is substantially free of (e.g., completely free of) sorbitol. In yet another example, the formulation is substantially free of (e.g., completely free of) trehalose.

### D. Delivery Devices Comprising High Concentration Antibody Formulations

Any of the high concentration antibody formulation disclosed herein may be placed in a delivery device, for example, a glass vial, a syringe, a pre-filled syringe, a pen delivery device, or an autoinjector. In some embodiments, the high concentration antibody formulation (e.g., comprising or consisting essentially of about 150 mg/ml of an anti-IgE antibody such as FB825, about 10 mM of the histidine, about 1.25% of the amino acid, about 75 mM of the sodium chloride, and about 0.01% of the polysorbate 80) can be contained in a pre-filled syringe. In some examples, the pre-filled syringe is a single-dose pre-filled syringe. In other examples, the high concentration antibody formulation is contained in an autoinjector. In yet other examples, the high concentration antibody formulation is contained in a pen delivery device (e.g., a pre-filled pen).

In some embodiments, a high concentration antibody formulation as disclosed herein can be delivered, e.g., subcutaneously, with a standard needle and syringe. In some embodiments, the syringe is a pre-filled syringe. In some embodiments, a pen delivery device or autoinjector is used to deliver the high concentration antibody formulation (e.g., for subcutaneous delivery). A pen delivery device can be reusable or disposable. Typically, a reusable pen delivery device utilizes a replaceable cartridge that contains the high concentration antibody formulation. Once the antibody formulation within the cartridge has been administered and the cartridge is empty, the empty cartridge can readily be discarded and replaced with a new cartridge that contains the antibody formulation. The pen delivery device can then be reused. In a disposable pen delivery device, there is no replaceable cartridge. Rather, the disposable pen delivery device comes prefilled with the antibody formulation held in a reservoir within the device. Once the reservoir is emptied of the antibody formulation, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BDM pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany). Examples of disposable pen delivery devices having applications in subcutaneous delivery of a high concentration antibody formulation disclosed herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN™ (Dey, L.P.), and the HUMIRA™ Pen (Abbott Labs, Abbott Park Ill.).

In some embodiments, the high concentration antibody formulation as disclosed herein is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

Any of the delivery devices containing the high concentration antibody formulation as disclosed herein is also within the scope of the present disclosure.

### II. Therapeutic Applications of High Concentration Antibody Formulations

To practice the method disclosed herein, an effective amount of any of the high concentration anti-IgE antibody formulations disclosed herein can be administered to a subject (e.g., a human) in need of the treatment via a suitable route, such as subcutaneous injection or intramuscular injection.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a disorder associated with IgE (e.g., allergic asthma, as well as other disorders known in the art and/or disclosed herein). A subject having an IgE-associated disorder such as allergic asthma can be identified by routine medical examination, e.g., laboratory tests. A subject suspected of having the IgE-associated disorder might show one or more symptoms of the disorder, e.g., elevated levels of IgE and/or hyper-reactivity to an allergen and/or antigen. A subject at risk for the disorder can be a subject having one or more of the risk factors for that disorder.

Exemplary IgE-associated disorders include, but are not limited to, asthma, allergic rhinitis, hyper IgE syndrome, atopic dermatitis, cold-induced urticaria, chronic urticaria, cholinergic urticaria, chronic rhinosinusitis, systemic mastocytosis, cutaneous mastocytosis, allergic bronchopulmonary aspergillosis, recurrent idiopathic angioedema, and interstitial cystitis, eosinophil-associated gastrointestinal disorders, a food allergy, or a drug allergy. In some examples, the target IgE-associated disorder is atopic dermatitis.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a disorder associated with IgE. Alternatively, sustained continuous release formulations of an anti-CεmX antibody may be appropriate. Various formulations and devices for achieving sustained release are known in the art.

In one example, dosages for an anti-CεmX antibody as described herein may be determined empirically in individuals who have been given one or more administration(s) of an anti-CεmX antibody. Individuals are given incremental dosages of the anti-CεmX antibody. To assess efficacy of the anti-CεmX antibody, an indicator of a disorder associated with IgE (such as levels of IgE) can be followed.

For the purpose of the present disclosure, the appropriate dosage of an anti-CεmX antibody will depend on the specific anti-CεmX antibody(s) (or compositions thereof) employed, the type and severity of disorder associated with IgE, whether the antibody is administered for preventive or therapeutic purposes, previous therapy, the patient's clinical history and response to the antibody, and the discretion of the attending physician. Typically the clinician will administer an anti-CεmX antibody, such as FB825, until a dosage is reached that achieves the desired result. Administration of an anti-CεmX antibody can be continuous or intermittent, depending, for example, upon the recipient's physiological condition, whether the purpose of the administration is therapeutic or prophylactic, and other factors known to skilled practitioners.

In some embodiments, the anti-CεmX antibody (e.g., FB825) described herein is administered to a subject in need of the treatment at an amount sufficient to reduce the level of the total IgE level by at least 20% (e.g., 30%, 40%, 50%, 60%, 70%, 80%, 90% or greater).

As used herein, the term “treating” refers to the application or administration of a composition including one or more active agents to a subject, who has a disease associated with IgE, a symptom of a disease associated with IgE, or a predisposition toward the disease, with the purpose to cure, heal, alleviate, relieve, alter, remedy, ameliorate, improve, or affect the disorder, the symptom of the disease, or the predisposition toward the disease.

Alleviating a disease associated with IgE includes delaying the development or progression of the disease, or reducing disease severity. Alleviating the disease does not necessarily require curative results. As used therein, “delaying” the development of a disease (such as a disease associated with IgE) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with IgE includes initial onset and/or recurrence.

To perform the methods as described herein, any of the anti-CεmX antibodies such as FB825 may be given to a subject in need of the treatment (e.g., a human patient) by a single dose or by multiple doses via a suitable route, for example, intravenous infusion or subcutaneous injection. The dosage of the anti-CεmX antibody for each administration may range from about 0.5 mg/kg to about 25 mg/kg (e.g., about 1 mg/kg to about 20 mg/kg, about 5 mg/kg to about 15 mg/kg, or about 10 mg/kg to about 20 mg/kg), depending upon various factors, including those described herein. For repeated administrations over several days or longer, depending on the condition, the treatment is sustained until a desired suppression of symptoms occurs or until sufficient therapeutic levels are achieved to alleviate a disorder associated with IgE, or a symptom thereof.

The administration of an anti-CεmX antibody (e.g., FB825) may be essentially continuous over a preselected period of time or may be in a series of spaced dose, e.g., either before, during, or after developing a disorder associated with IgE. An exemplary dosing regimen comprises administering to a subject in need of the treatment a first dose of an anti-CεmX antibody (e.g., at 3 mg/kg, 5 mg/kg, 10 mg/kg, 15 mg/kg, 20 mg/kg, or 25 mg/kg), followed by a second dose of the antibody at least 3 months after the first dose (e.g., 4 months, 5 months, or 6 months). The dosage of the second administration may be higher, the same, or lower than the first administration. Other dosage regimens may be useful depending upon the pattern of pharmacokinetic decay that a practitioner wishes to achieve.

In some embodiments, a subject in need of the treatment (e.g., a human patient having an IgE-associated allergic disorder such as atopic dermatitis) can be given a first dose of the antibody at a suitable amount (e.g., at 3 mg/kg, 5 mg/kg, 10 mg/kg, 15 mg/kg, 20 mg/kg, or 25 mg/kg). The subject is then monitored periodically for symptoms indicative of an IgE-associated disorder, for example, allergic reactions and/or an elevated level of total IgE. A second dose of the antibody may be given to the subject when such a symptom is observed. The first dose and the second dose may be administered to the subject at least three months apart, for example, 3-month apart, 4-month apart, 5-month apart, 6-month apart, 9-month apart, or 12-month apart.

In some embodiments, a subject in need of the treatment (e.g., a human patient having an IgE-associated allergic disorder such as atopic dermatitis) is given only one dose of the high concentration antibody formulation. The antibody formulation may comprise the anti-IgE antibody of FB825.

Also within the scope of the present disclosure are preventive treatments of an IgE-associated disorder with any of the anti-CεmX antibodies to reduce the risk for occurrence of such a disorder. Subjects suitable for such a preventive treatment may be human patients having history of an IgE-associated disorder and/or family history of an IgE-associated disorder.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

The particular dosage regimen, i.e., dose, timing and repetition, used in the method described herein will depend on the particular subject and that subject's medical history. Any of the anti-CεmX antibodies described herein may be used in conjunction with other agents (e.g., other agents for treating IgE-associated disorders) that serve to enhance and/or complement the effectiveness of the agents.

In some embodiments, an anti-CεmX antibody as described herein, for example, FB825, is used for treating atopic dermatitis as follows. Atopic dermatitis, also known as eczema, is a chronical skin condition characterized by redness and/or itchy. It is common in children but can occur at any age. A patient who needs the treatment can be identified by routine medical practice as having one or more symptoms of atopic dermatitis, including dry skin, itching, red to brownish-gray patches, small, raised bumps, which may leak fluid and crust over when scratched, thickened, cracked, scaly skin, and/or raw, sensitive, swollen skin from scratching. In some instances, the total IgE level and the level of allergen-specific IgE of a candidate subject can be examined via routine practice. If the IgE level of the candidate subject (e.g., the total IgE, the allergen-specific IgE, or both) is higher than a normal level (representing the average IgE level in subjects of the same species, e.g., humans, who are free of atopic dermatitis or other allergic disorders associated with IgE).

A human patient who needs the treatment may be given a first a dose of the antibody, which may range from 3 mg/kg to 8 mg/kg, via a conventional route as described herein. In some instances, the first dose is 5 mg/kg. After the first dose, the total IgE level of the patient can be monitored. If the reduction of the IgE level 3-4 weeks after the first dose is less than 50%, a second dose of the antibody may be given to the patient 3-4 weeks after the first dose. The second dose may be identical to the first dose, or lower than the first dose. In some instances, both the first dose and the second dose are 5 mg/kg and are administered via IV infusion in a 1-2 hour period. Other biomarkers indicating efficacy and/or safety could also be monitored during the course of the treatment. Such biomarkers include, but are not limited to, thymus and activation regulated chemokine (TARC), Eotaxin-3, thymic stromal lymphopoietin (TSLP), periostin, IL-la, IL-4, IL-5, IL-13, IL-16, IL-31, M-CSF, or a combination thereof.

The human patient subject to the above-noted treatment may have chronic atopic dermatitis for at least 3 years as diagnosed by routine medical practice, for example, defined by the Eishenfield revised criteria of Hannifin and Rajka and supported by positive allergen-specific IgE. The patient may have one or more of the following features: (i) eczema area and severity index (EASI) score greater than 14, (ii) Investigator's Global Assessment (IGA) score greater than 3 (5-point scale), (iii) greater than 10% body surface area (BSA), (iv) history of inadequate response to a stable regimen of topical corticosteroids or calcineurin inhibitors for at least one month or at least three months before the treatment. Further, the human patient may be given stable doses of emollient twice daily for at least 7 days before the treatment.

In some embodiments, the subject for the treatment disclosed herein is a human patient having a low IgG4 level (e.g., low serum IgG4 level). If desired, IgE levels are measured along with IgG4 levels. The human patient may have or suspected of having an IgE-associated allergic disorder such as atopic dermatitis.

IgG4 in bodily fluids such as serum can be detected using methods known in the art, e.g., quantitative assays discussed in WO2019/089978, including an enzyme-linked immunosorbent assay (ELISA); an alkaline phosphatase immunoassay auto-analyzer, such as an EVEVIULITER system (Siemens Healthcare Diagnostics, Erlangen, Germany); a radioallergosorbent test (RAST), or a fluoroenzyme immunoassay auto-analyzer, such as the ImmunoCAP® system (Thermo Fisher Scientific/Phadia, Uppsala, Sweden). Additional suitable methods include a fluorescence enzyme immunoassay (FEIA) auto-analyzer (e.g., ImmunoCAPR system). Another technique may be used as the level of antibody (e.g., IgE or IgG4) determined by that technique may be normalized to a measurement by a fluorescence enzyme immunoassay auto-analyzer. That is, a level of antibody (e.g., IgE or lgG4) can be determined by a technique, and can correspond to a level as measured by a fluorescence enzyme immunoassay auto-analyzer. The level of the biomarker is preferably determined in vitro.

The IgG4 level obtained from a patient candidate may be compared with a predetermined value, which represents the IgG4 level that distinguishes patients responsive to an anti-IgE therapy relative to those that have poor responsiveness to the therapy. Such a predetermined value may be set forth based on analysis of representative IgG4 levels in anti-IgE therapy responders versus anti-IgE therapy non-responders. The predetermined value may take into consideration matched physiological features as the subject, for example, age, gender, ethnic background, etc.

In some instances, the anti-CεmX antibody as described herein (e.g., FB825) may be co-used with moisturizers (e.g., at stable doses such as at least twice daily) and/or topical corticosteroid (TCS). A medium potency TCS may be applied to areas with active lesions and may switch to low potency TCS after the lesions are under control. If lesions reoccur, treatment with a medium potency TCS may resume with a step-down approach. If lesions are persisting or worsening after daily treatment with a medium potency TCS, a high or super-high potency TCS may be used, unless it is deemed unsafe. A low potency TCS may be used on areas of thin skin (e.g., face, neck, intertriginous, genital areas, or areas of skin atrophy) or on areas where continued use of medium potency TCS is considered unsafe.

TCS having low, medium, and high or super-high potency is well known in the art. Exemplary medium potency TCS includes 0.05% fluticasone propionate cream, 0.1% mometasone furoate cream, or 0.06% betamethasone valerate cream. Exemplary low potency TCS includes 1% hydrocortisone ointment. Exemplary high potency TCS can be 0.05% fluocinonide cream or 0.25% desomimetasone ointment. Exemplary super-high potency TCS can be 0.05% clobetasol propionate ointment.

In some instances, the patient subject to the treatment described herein is free of one or more of the following therapy: (i) topical tacrolimus and pimecrolimus, (ii) systemic treatment of corticosteroids, (iii) leukotriene inhibitors, (iv) allergen immunotherapy, (v) systemic treatment of immunosuppressors or immunomodulators (e.g., cyclosporine, mycophenolate-mofetil, IFN-γ, azathioprine, methotrexate, or biologics), (vi) live (e.g., attenuated) vaccines, and/or (vii) traditional Chinese medicine. The patient may also be free of any surgical procedures and/or UV procedures.

Any of the methods described herein may further comprise assessing occurrence of decreased hemoglobin, upper respiratory tract infection, urinary tract infection, or a combination thereof in the subject after the first dose. If one or more occurrences are observed, the amount of the anti-CεmX antibody (e.g., FB825) of the second dose may be reduced. Alternatively, the treatment may be stopped.

### III. Kits for Use in Treating IgE-Associated Disorders

The present disclosure also provides kits for use in treating IgE-associated disorders with any of the high concentration antibody formulations disclosed herein. Such kits can include one or more containers comprising the high concentration antibody formulation (e.g., a formulation comprising about 150 mg/ml of the antibody such as FB825, about 10 mM of the histidine, about 1.25% of the amino acid, about 75 mM of the sodium chloride, and about 0.01% of the polysorbate 80).

In some embodiments, the kit can comprise instructions for use in accordance with any of the methods described herein. The included instructions can comprise a description of administration of the high concentration antibody formulation to treat, delay the onset, or alleviate an IgE-associated disorder according to any of the methods described herein. The kit may further comprise a description of selecting an individual suitable for treatment based on identifying whether that individual has, is suspected of having, or is at risk for the disorder. In still other embodiments, the instructions comprise a description of administering the high concentration antibody formulation to a subject in need of the treatment to reduce the risk for developing the IgE-associated disorder.

The instructions relating to the use of the high concentration antibody formulation generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating an IgE-associated disorder. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump.

Kits may optionally provide additional components such as buffers and interpretive information. Normally, the kit comprises a container and a label or package insert(s) on or associated with the container. In some embodiments, the present disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Screening for Suitable Surfactant

FB825 was concentrated to 10 mg/ml using a 10 kDa MWCO centrifugal concentrator and then spiked with various candidate surfactants to a final desired concentration. The resultant formulations were subjected to agitation for 4 hours at room temperature and then analysed by SEC-HPLC and Micro-Flow Imaging (MFI). Size measurements and counts of subvisible particles were collected using an MFI instrument from Brightwell Technologies. Digital filters were applied to the MFI data to exclude air bubbles or static particles.

Three surfactants, polysorbate 80 (P80, 0.01%), polysorbate 20 (P20, 0.01%), and Pluronic F68 (F68, 0.1%) were investigated in this study. The MFI analysis showed that the agitated samples with no surfactant showed accumulation of particles, while addition of the surfactant reduced particle accumulation. Polysorbate 80 at 0.01% showed the lowest particle counts.

### Example 2: Screening for Suitable Buffer and Tonicity Modifier

Multiple buffer agents (Acetate, Histidine and Phosphate), pH conditions (pH 4.0, 5.0, 6.0, 7.0 and 8.0), and tonicity modifiers (sodium chloride and sorbitol) were investigated in this Example to identify the option components and concentrations for producing a stable FB825 formulation. Table 1 below lists components in various F825 formulations tested in this example.

TABLE 1
Tested FB825 Formulations

| Formu- | | | Tonicity | Surfactant |
|---|---|---|---|---|
| lation | Buffer | pH | Modifier | PS80 (%) |
| A4N | 10 mM Sodium Acetate | 4.0 | 150 mM NaCl | 0.01 |
| A4S | 10 mM Sodium Acetate | 4.0 | 5% Sorbitol | 0.01 |
| A5N | 10 mM Sodium Acetate | 5.0 | 150 mM NaCl | 0.01 |
| A5S | 10 mM Sodium Acetate | 5.0 | 5% Sorbitol | 0.01 |
| H6N | 10 mM Histidine | 6.0 | 150 mM NaCl | 0.01 |
| H6S | 10 mM Histidine | 6.0 | 5% Sorbitol | 0.01 |
| P6N | 10 mM Sodium Phosphate | 6.0 | 150 mM NaCl | 0.01 |
| P6S | 10 mM Sodium Phosphate | 6.0 | 5% Sorbitol | 0.01 |
| P7N | 10 mM Sodium Phosphate | 7.0 | 150 mM NaCl | 0.01 |
| P7S | 10 mM Sodium Phosphate | 7.0 | 5% Sorbitol | 0.01 |
| P8N | 10 mM Sodium Phosphate | 8.0 | 150 mM NaCl | 0.01 |
| P8S | 10 mM Sodium Phosphate | 8.0 | 5% Sorbitol | 0.01 |

The formulations listed in Table 1 above were examined for their stability under agitation, freeze/thaw, photosensitivity, and temperature conditions provided in Table 2 below using SEC-HPLC analysis.

TABLE 2
Conditions for Stability Tests
Stress conditions and analysis time points

| Stress | Conditions | Time Point (s) |
|---|---|---|
| Agitation | Vortex | 4 hours |
| Freeze/thaw | −70° C. to ambient | 5 consecutive cycles |
| | temperature | |
| Photosensitivity | Broad spectrum UV light | 24 hours |
| | exposure | |
| Temperature | −20° C.; 5° C.; 25° C.; | 0, 1, 2, 4, 8 weeks |
| | 40° C. | |

The results show that the formulation (H6N) containing 10 mM Histidine, 150 mM NaCl, 0.01% PS80 at pH 6.0 exhibited the best stability under the tested conditions noted above. For example, Formulation H6N showed the best stability after being kept at 40° C. for 8 weeks. Table 3.

TABLE 3
Stability Test - Temperature (40° C. for 8 weeks)

| | | SEC-HPLC | | | SCX-HPLC | | |
|---|---|---|---|---|---|---|---|
| | | analysis | | | analysis | | |
| | | | Main | | Acid | Main | Basic |
| | | HMW | Peak | LMW | Peak | Peak | Peak |
| Stress | Formulation | (%) | (%) | (%) | (%) | (%) | (%) |
| 40° C. for | DS control | 0.3 | 98.9 | 0.8 | 27.2 | 61.1 | 11.6 |
| 8 weeks | A4N | 0.0 | 19.2 | 80.7 | 60.1 | 13.4 | 24.1 |
| | A4S | 0.1 | 76.3 | 19.5 | 58.4 | 19.4 | 21.7 |
| | A5N | 0.6 | 86.8 | 12.2 | 51.4 | 27.8 | 20.8 |
| | A5S | 0.5 | 93.6 | 5.3 | 57.9 | 27.7 | 14.3 |
| | H6N | 0.6 | 96.2 | 3.3 | 50.1 | 37.5 | 12.4 |
| | H6S | 0.5 | 96.0 | 3.5 | 57.3 | 30.5 | 12.2 |
| | P6N | 0.6 | 95.1 | 4.3 | 54.4 | 33.6 | 11.9 |
| | P6S | 0.6 | 96.1 | 3.4 | 61.6 | 28.0 | 10.4 |
| | P7N | 0.9 | 94.9 | 4.1 | 76.3 | 15.3 | 8.2 |
| | P7S | 0.9 | 95.2 | 3.8 | 76.0 | 15.2 | 8.8 |
| | P8N | 1.3 | 93.9 | 4.8 | 88.9 | 6.0 | 4.7 |
| | P8S | 1.1 | 94.5 | 4.4 | 82.6 | 11.8 | 5.6 |

### Example 3: Screening for Amino Acid Excipient, Stabilizer, and pH Conditions

FB825 was concentrated to about 200 mg/ml, using a 10 kDa MWCO centrifugal concentrator (20 mg/ml of stock FB825 in 20 mM Histidine and 140 mM NaCl, 0.02% PS80 at pH 6.5). The concentrated FB825 formulations were dialyzed against 10 mM Histidine, 75 mM NaCl (pH 6.0), and 0.01% PS80 or 10 mM Histidine (pH 6.0) and 0.01% PS80 in screening for stabilizer and pH effect). The dialyzed samples were concentrated to about 200 mg/ml FB825 and used for identifying optional amino acid excipients, stabilizers and pH conditions.

### (i) Screening for Amino Acid Excipients

FB825 formulations each comprising one of the 19 amino acids listed in Table 5 below were examined for physical features also listed in Table 4 after the formulations were kept at 50° ° C. for 48 hours.

TABLE 4
Conditions for Amino Acid Excipient Screening
19 Different amino acid excipients

| Time & Temp. | Analytical Method |
|---|---|
| 50° C., 48 h | Turbidity (A₆₅₀) |
| | Osmolality |
| | SEC-HPLC |
| | DLS |

The results are shown in Table 5 below.

TABLE 5
Amino Acid Excipient Screening

| Amino | | | | | | | | Main | |
|---|---|---|---|---|---|---|---|---|---|
| Acid | | | Tonicity | | FB825 | Turbidity | HMW | Peak | LMW |
| (w/v) | pH | Buffer | Modifier | Surfactant | (mg/ml) | (A₆₅₀) | (%) | (%) | (%) |
| None | 6.0 | 10 mM | 75 mM | 0.01% PS80 | 10 | NA | 0.5 | 99.5 | 0.0 |
| | | Histidine | NaCl | | | | | | |
| None | 6.0 | 10 mM | 75 mM | 0.01% PS80 | 10 | NA | 0.5 | 99.5 | 0.0 |
| | | Histidine | NaCl | | | | | | |
| None | 6.0 | 10 mM | 75 mM | 0.01% PS80 | 150 | NA | 0.9 | 99.1 | 0.0 |
| | | Histidine | NaCl | | | | | | |
| None | 6.0 | 10 mM | 75 mM | 0.01% PS80 | 150 | 0.001 | 1.8 | 98.1 | 0.1 |
| | | Histidine | NaCl | | | | | | |
| 1.25% | 6.0 | 10 mM | 75 mM | 0.01% PS80 | 150 | 0.004 | 1.7 | 98.2 | 0.1 |
| L-Ala | | Histidine | NaCl | | | | | | |
| 1.25% | 6.0 | 10 mM | 75 mM | 0.01% PS80 | 150 | 0.003 | 1.4 | 98.5 | 0.1 |
| L-Arg | | Histidine | NaCl | | | | | | |
| 0.75% | 6.0 | 10 mM | 75 mM | 0.01% PS80 | 150 | 0.006 | 1.5 | 98.4 | 0.1 |
| L-Asn | | Histidine | NaCl | | | | | | |
| 0.2% | 6.0 | 10 mM | 75 mM | 0.01% PS80 | 150 | 0.003 | 2.0 | 97.9 | 0.1 |
| L-Asp | | Histidine | NaCl | | | | | | |
| 0.5% | 6.0 | 10 mM | 75 mM | 0.01% PS80 | 150 | 0.003 | 1.9 | 98.0 | 0.1 |
| L-Gln | | Histidine | NaCl | | | | | | |
| 0.225% | 6.0 | 10 mM | 75 mM | 0.01% PS80 | 150 | 0.004 | 1.8 | 98.1 | 0.2 |
| L-Glu | | Histidine | NaCl | | | | | | |
| 1.25% | 6.0 | 10 mM | 75 mM | 0.01% PS80 | 150 | 0.003 | 1.4 | 98.5 | 0.1 |
| L-Gly | | Histidine | NaCl | | | | | | |
| 1.0% | 6.0 | 10 mM | 75 mM | 0.01% PS80 | 150 | 0.003 | 1.7 | 98.1 | 0.3 |
| L-His | | Histidine | NaCl | | | | | | |
| 0.75% | 6.0 | 10 mM | 75 mM | 0.01% PS80 | 150 | 0.002 | 1.6 | 98.3 | 0.1 |
| L-Ile | | Histidine | NaCl | | | | | | |
| 0.5% | 6.0 | 10 mM | 75 mM | 0.01% PS80 | 150 | 0.002 | 1.7 | 98.2 | 0.2 |
| L-Leu | | Histidine | NaCl | | | | | | |
| 1.25% | 6.0 | 10 mM | 75 mM | 0.01% PS80 | 150 | 0.002 | 1.4 | 98.5 | 0.1 |
| L-Lys | | Histidine | NaCl | | | | | | |
| 0.75% | 6.0 | 10 mM | 75 mM | 0.01% PS80 | 150 | 0.002 | 1.5 | 98.3 | 0.1 |
| L-Met | | Histidine | NaCl | | | | | | |
| 0.625% | 6.0 | 10 mM | 75 mM | 0.01% PS80 | 150 | 0.003 | 1.6 | 98.3 | 0.1 |
| L-Phe | | Histidine | NaCl | | | | | | |
| 1.25% | 6.0 | 10 mM | 75 mM | 0.01% PS80 | 150 | 0.003 | 1.7 | 98.2 | 0.1 |
| L-Pro | | Histidine | NaCl | | | | | | |
| 1.25% | 6.0 | 10 mM | 75 mM | 0.01% PS80 | 150 | 0.002 | 1.4 | 98.4 | 0.1 |
| L-Ser | | Histidine | NaCl | | | | | | |
| 1.25% | 6.0 | 10 mM | 75 mM | 0.01% PS80 | 150 | 0.002 | 1.4 | 98.5 | 0.1 |
| L-Thr | | Histidine | NaCl | | | | | | |
| 0.25% | 6.0 | 10 mM | 75 mM | 0.01% PS80 | 150 | 0.003 | 1.6 | 98.3 | 0.1 |
| L-Trp | | Histidine | NaCl | | | | | | |
| 0.0125% | 6.0 | 10 mM | 75 mM | 0.01% PS80 | 150 | 0.002 | 1.7 | 98.1 | 0.1 |
| L-Tyr | | Histidine | NaCl | | | | | | |
| 1.25% | 6.0 | 10 mM | 75 mM | 0.01% PS80 | 150 | 0.002 | 1.5 | 98.3 | 0.1 |
| L-Val | | Histidine | NaCl | | | | | | |

As shown in Table 4 above, arginine, glycine, lysine, serine, and threonine demonstrated superior physical stability in this assay.

### (b) Screening for Stabilizer and pH Conditions

The formulations listed in Table 7 below, comprising various stabilizers and pH conditions, were examined for the physical stability features listed in Table 6 below. The results are shown in Tables 7 and 8.

TABLE 6
Conditions for Stabilizer and pH Condition Screening
Stabilizer and pH effect

| Time & Temp. | Analytical Method |
|---|---|
| 55° C., 48 h | Turbidity (A₆₅₀) |
| | Osmolality |
| | SEC-HPLC |

TABLE 7
Stabilizer and pH Condition Screening - SEC-HPLC

| | Amino | | | | | | | SEC-HPLC |
|---|---|---|---|---|---|---|---|---|
| Form. | Acid | Stabilizer | | | Tonicity | | FB825 | Main peak |
| Code | (w/v) | (w/v) | pH | Buffer | Modifier | Surfactant | (mg/ml) | (%) |
| C20 | None | None | 6 | 10 mM | 75 mM | 0.01% PS80 | 20 | 98.8 |
| | | | | Histidine | NaCl | | | |
| C150 | None | None | 6 | 10 mM | 75 mM | 0.01% PS80 | 150 | 97.5 |
| | | | | Histidine | NaCl | | | |
| R5.5 | 1.25% | None | 5.5 | 10 mM | 75 mM | 0.01% PS80 | 150 | 98.1 |
| | L-Arg | | | Histidine | NaCl | | | |
| G5.5 | 1.25% | None | 5.5 | 10 mM | 75 mM | 0.01% PS80 | 150 | 97.8 |
| | L-Gly | | | Histidine | NaCl | | | |
| K5.5 | 1.25% | None | 5.5 | 10 mM | 75 mM | 0.01% PS80 | 150 | 97.8 |
| | L-Lys | | | Histidine | NaCl | | | |
| S5.5 | 1.25% | None | 5.5 | 10 mM | 75 mM | 0.01% PS80 | 150 | 97.8 |
| | L-Ser | | | Histidine | NaCl | | | |
| T5.5 | 1.25% | None | 5.5 | 10 mM | 75 mM | 0.01% PS80 | 150 | 97.8 |
| | L-Thr | | | Histidine | NaCl | | | |
| R6.0 | 1.25% | None | 6.0 | 10 mM | 75 mM | 0.01% PS80 | 150 | 97.9 |
| | L-Arg | | | Histidine | NaCl | | | |
| G6.0 | 1.25% | None | 6.0 | 10 mM | 75 mM | 0.01% PS80 | 150 | 97.8 |
| | L-Gly | | | Histidine | NaCl | | | |
| K6.0 | 1.25% | None | 6.0 | 10 mM | 75 mM | 0.01% PS80 | 150 | 97.8 |
| | L-Lys | | | Histidine | NaCl | | | |
| S6.0 | 1.25% | None | 6.0 | 10 mM | 75 mM | 0.01% PS80 | 150 | 97.8 |
| | L-Ser | | | Histidine | NaCl | | | |
| T6.0 | 1.25% | None | 6.0 | 10 mM | 75 mM | 0.01% PS80 | 150 | 98.0 |
| | L-Thr | | | Histidine | NaCl | | | |
| R6.5 | 1.25% | None | 6.5 | 10 mM | 75 mM | 0.01% PS80 | 150 | 97.9 |
| | L-Arg | | | Histidine | NaCl | | | |
| G6.5 | 1.25% | None | 6.5 | 10 mM | 75 mM | 0.01% PS80 | 150 | 97.7 |
| | L-Gly | | | Histidine | NaCl | | | |
| K6.5 | 1.25% | None | 6.5 | 10 mM | 75 mM | 0.01% PS80 | 150 | 97.7 |
| | L-Lys | | | Histidine | NaCl | | | |
| S6.5 | 1.25% | None | 6.5 | 10 mM | 75 mM | 0.01% PS80 | 150 | 97.7 |
| | L-Ser | | | Histidine | NaCl | | | |
| T6.5 | 1.25% | None | 6.5 | 10 mM | 75 mM | 0.01% PS80 | 150 | 97.7 |
| | L-Thr | | | Histidine | NaCl | | | |
| TT6.0 | 2.5% | None | 6.0 | 10 mM | None | 0.01% PS80 | 150 | 97.8 |
| | L-Thr | | | Histidine | | | | |
| Suc5.5 | None | 5% | 5.5 | 10 mM | None | 0.01% PS80 | 150 | 97.3 |
| | | Sucrose | | Histidine | | | | |
| Sor5.5 | None | 2.5% | 5.5 | 10 mM | None | 0.01% PS80 | 150 | 97.3 |
| | | Sorbitol | | Histidine | | | | |
| Tre5.5 | None | 5% | 5.5 | 10 mM | None | 0.01% PS80 | 150 | 97.0 |
| | | Trehalose | | Histidine | | | | |
| Suc6.0 | None | 5% | 6.0 | 10 mM | None | 0.01% PS80 | 150 | 97.1 |
| | | Sucrose | | Histidine | | | | |
| Sor6.0 | None | 2.5% | 6.0 | 10 mM | None | 0.01% PS80 | 150 | 97.0 |
| | | Sorbitol | | Histidine | | | | |
| Tre6.0 | None | 5% | 6.0 | 10 mM | None | 0.01% PS80 | 150 | 97.1 |
| | | Trehalose | | Histidine | | | | |
| Suc6.5 | None | 5% | 6.5 | 10 mM | None | 0.01% PS80 | 150 | 96.9 |
| | | Sucrose | | Histidine | | | | |
| Sor6.5 | None | 2.5% | 6.5 | 10 mM | None | 0.01% PS80 | 150 | 96.8 |
| | | Sorbitol | | Histidine | | | | |
| Tre6.5 | None | 5% | 6.5 | 10 mM | None | 0.01% PS80 | 150 | 96.7 |
| | | Trehalose | | Histidine | | | | |

In this study, formulations containing the tested stabilizer (sucrose, sorbitol, or trehalose) showed relatively lower main peak percentages as compared with the corresponding non-stabilizer formulations. Formulations R5.5, T6.0, and R6.5 showed the highest main peak percentages, indicating high stability after being incubated at 55° C. for 48 hours. All arginine-containing formulations showed both high main peak percentages and low HMW peak percentages, indicating stability (e.g., little degradation and aggregation) of these formulations.

TABLE 8
Stabilizer and pH Condition Screening - Osmolality

| Form. | Theoretical | Measured | Form. | Theoretical | Measured |
|---|---|---|---|---|---|
| Code | mOsm | mOsm | Code | mOsm | mOsm |
| C20 | 180 | 155 | K6.5 | 378 | 348 |
| C150 | 310 | 311 | S6.5 | 405 | 365 |
| R5.5 | 367 | 364 | T6.5 | 394 | 354 |
| G5.5 | 443 | 404 | TT6.0 | 369 | 335 |
| K5.5 | 378 | 356 | Suc5.5 | 306 | 279 |
| S5.5 | 405 | 362 | Sor5.5 | 297 | 240 |
| T5.5 | 394 | 353 | Tre5.5 | 292 | 255 |
| R6.0 | 367 | 377 | Suc6.0 | 306 | 253 |
| G6.0 | 443 | 392 | Sor6.0 | 297 | 270 |
| K6.0 | 378 | 351 | Tre6.0 | 292 | 265 |
| S6.0 | 405 | 353 | Suc6.5 | 306 | 274 |
| T6.0 | 394 | 337 | Sor6.5 | 297 | 243 |
| R6.5 | 367 | 364 | Tre6.5 | 292 | 254 |
| G6.5 | 443 | 414 | | | |

All formulations displayed osmolality values comparable to theoretical values. Most of the formulations were near isotonic, with slight variations.

### (c) Stress Optimization Screening

High concentration FB825 formulations (˜150 mg/ml) were stored at 45° ° C. up to 2 weeks or stored at 55° ° C. for 1 week. These samples were then analyzed by SEC-HPLC. As shown in Table 9 below, the formulations stored at 45° ° C. up to 2 weeks showed no significant changes in main peak values, while the formulations stored at 55° ° C. for 1 week showed a greater decrease in main peak percentage (˜4%).

TABLE 9
Stress Optimization Screening

| | Incubation | HMW | Main Peak | LMW |
|---|---|---|---|---|
| Sample | (° C.) | (%) | (%) | (%) |
| C150 T = 0 | NA | 2.1 | 95.4 | 2.4 |
| C150 T = 1 wk, 45° C. | 45° C. | 3.1 | 93.3 | 3.6 |
| C150 T = 2 wk, 45° C. | 45° C. | 3.6 | 93.4 | 3.0 |
| C150 T = 1 wk, 55° C. | 55° C. | 4.8 | 91.5 | 3.8 |

In addition, the formulations listed in Table 10 were subject to the same stability analysis after being kept at 55° C. for one week and the results are shown in Table 11.

TABLE 10
Formulations for Stress Optimization Screening

| | Amino | | | | | |
|---|---|---|---|---|---|---|
| Form. | Acid | | | Tonicity | | FB825 |
| Code | (w/v) | pH | Buffer | Modifier | Surfactant | (mg/ml) |
| C20 | None | 6.0 | 10 mM | 75 mM | 0.01% PS80 | 20 |
| | | | Histidine | NaCl | | |
| C150 | None | 6.0 | 10 mM | 75 mM | 0.01% PS80 | 150 |
| | | | Histidine | NaCl | | |
| R5.5 | 1.25% | 5.5 | 10 mM | 75 mM | 0.01% PS80 | 150 |
| | L-Arg | | Histidine | NaCl | | |
| G5.5 | 1.25% | 5.5 | 10 mM | 75 mM | 0.01% PS80 | 150 |
| | L-Gly | | Histidine | NaCl | | |
| K5.5 | 1.25% | 5.5 | 10 mM | 75 mM | 0.01% PS80 | 150 |
| | L-Lys | | Histidine | NaCl | | |
| S5.5 | 1.25% | 5.5 | 10 mM | 75 mM | 0.01% PS80 | 150 |
| | L-Ser | | Histidine | NaCl | | |
| T5.5 | 1.25% | 5.5 | 10 mM | 75 mM | 0.01% PS80 | 150 |
| | L-Thr | | Histidine | NaCl | | |
| R6.0 | 1.25% | 6.0 | 10 mM | 75 mM | 0.01% PS80 | 150 |
| | L-Arg | | Histidine | NaCl | | |
| G6.0 | 1.25% | 6.0 | 10 mM | 75 mM | 0.01% PS80 | 150 |
| | L-Gly | | Histidine | NaCl | | |
| K6.0 | 1.25% | 6.0 | 10 mM | 75 mM | 0.01% PS80 | 150 |
| | L-Lys | | Histidine | NaCl | | |
| S6.0 | 1.25% | 6.0 | 10 mM | 75 mM | 0.01% PS80 | 150 |
| | L-Ser | | Histidine | NaCl | | |
| T6.0 | 1.25% | 6.0 | 10 mM | 75 mM | 0.01% PS80 | 150 |
| | L-Thr | | Histidine | NaCl | | |
| R6.5 | 1.25% | 6.5 | 10 mM | 75 mM | 0.01% PS80 | 150 |
| | L-Arg | | Histidine | NaCl | | |
| TT6.0 | 2.50% | 6.0 | 10 mM | None | 0.01% PS80 | 150 |
| | L-Thr | | Histidine | | | |
| RR6.0 | 2.50% | 6.0 | 10 mM | None | 0.01% PS80 | 150 |
| | L-Arg | | Histidine | | | |

TABLE 11
Stress Optimization Screening Results

| | Incubation | Turbidity | HMW | Main Peak | LMW |
|---|---|---|---|---|---|
| Form. Code | (55° C.) | (A₆₅₀) | (%) | (%) | (%) |
| C20 T = 0, 55° C. | NA | NA | 0.8 | 99.2 | 0.0 |
| C20 T = 1 wk, 55° C. | 55° C. | 0.001 | 1.4 | 97.6 | 1.0 |
| C150 T = 0, 55° C. | NA | NA | 0.8 | 99.2 | 0.0 |
| C150 T = 1 wk, 55° C. | 55° C. | 0.004 | 4.7 | 94.4 | 0.9 |
| R5.5 T = 1 wk, 55° C. | 55° C. | 0.008 | 3.0 | 96.2 | 0.9 |
| RR6.0 T = 1 wk, 55° C. | 55° C. | 0.003 | 3.3 | 95.7 | 1.0 |
| R6.0 T = 1 wk, 55° C. | 55° C. | 0.004 | 3.4 | 95.6 | 1.0 |
| K5.5 T = 1 wk, 55° C. | 55° C. | 0.004 | 3.6 | 95.5 | 1.0 |
| R6.5 T = 1 wk, 55° C. | 55° C. | 0.004 | 3.7 | 95.3 | 1.0 |
| K6.0 T = 1 wk, 55° C. | 55° C. | 0.003 | 3.8 | 95.3 | 0.9 |
| T5.5 T = 1 wk, 55° C. | 55° C. | 0.004 | 3.9 | 95.1 | 1.0 |
| G5.5 T = 1 wk, 55° C. | 55° C. | 0.003 | 4.0 | 95.1 | 1.0 |
| G6.0 T = 1 wk, 55° C. | 55° C. | 0.004 | 4.0 | 95.0 | 1.0 |
| S5.5 T = 1 wk, 55° C. | 55° C. | 0.004 | 4.4 | 94.5 | 1.0 |
| T6.0 T = 1 wk, 55° C. | 55° C. | 0.003 | 4.5 | 94.5 | 1.0 |
| S6.0 T = 1 wk, 55° C. | 55° C. | 0.004 | 4.7 | 94.4 | 1.0 |
| TT6.0 T = 1 wk, 55° C. | 55° C. | 0.004 | 5.0 | 94.0 | 1.1 |

As shown in Table 11, the arginine-containing formulations showed the highest stability in the stress optimization screening assay. The arginine-containing formulations (R5.5, R6.0, and RR6.0) were clear, displayed low turbidity values, and isotonic. These formulations also showed good injectable features in injectability studies.

### Example 4. Accelerated Stability Assessment of High Concentration Formulations

Formulations C150, R5.5, R6.0, and RR6.0 were further investigated in the accelerated stability assay under the conditions shown in Table 12.

TABLE 12
Conditions for Accelerated Stability Analysis

| Stress | Condition | Time Point(s) |
|---|---|---|
| Temperature | −20° C.; 5° C.; 25° C.; | 0, 2, 4, 8 weeks |
| | 40° C. | |
| Agitation | Vortex | 4 hours |
| Freeze/Thaw | −70° C. to ambient | 5 consecutive cycles |
| Photosensitivity | UV light exposure (broad | 8 watt hours/square |
| | spectrum) | meter |

SEC-HPLC analysis indicate that no significant changes were observed after the formulations were treated by agitation and freeze/thaws. Further, no significant differences were observed among the four formulations after US light exposures. In addition, Formulations R5.5, R6.0, and RR6.0 showed excellent physical stability and no significant differences in purity were observed among these three formulations.

### Example 5. Long-Term Stability Assessment

Formulations C10, R6.0, and RR6.0 were subject to long-term stability analysis as shown in Table 13 by visual inspection, protein concentration (A280), pH, osmolality, SEC-HPLC, SCX-HPLC, and SDS-PAGE.

TABLE 13
Long-Term Stability Assessment

| Stress | Conditions | | Time Point (s) |
|---|---|---|---|
| Temperature | −70° | C. | 1, 3, 6, 9, 12, 18 and 24 months |
| | 5° | C. | 0, 1, 3, 6, 9, 12, 18 and 24 months |
| | 25° | C. | 1, 3 and 6 months |
| | 40° | C. | 1 and 3 months |

SEC-HPLC method was performed under the following conditions:
Column: Tosoh TSK-Gel G3000W_{XL}, 7.8 mm ID×30 cm, 5 μm P/N 08541
Mobile Phase: 4× Dulbecco's PBS (pH 7.3)
Instrument: Agilent 1100 HPLC system
Flow rate: 0.4 ml/min
Column Temp: Ambient
Detection: 280 nm
Sample load: 20 μg (diluted with mobile phase)
Total run time: 30 minutes

The SCX-HPLC method was performed under the following conditions:
Column: Dinoex MabPac SCX-10, 4×250 mm, 10 μm P/N 074625
Mobile Phase A: 20 mM Tris (pH 7.2)
Mobile Phase A: 20 mM Tris and 0.2 M NaCl (pH 7.2)
Instrument: Agilent 1100 HPLC system
Flow rate: 0.5 ml/min
Column Temp: 40
Autosampler: 4
Detection: 215 nm
Sample load: 50 μg (diluted with mobile phase A)
Total run time: 90 minutes

Table 14 below shows the SEC-HPLC and SCX-HPLC results of Formulations C10, R6.0, and RR6.0 during a two-year period. High concentration formulations R6.0, and RR6.0 showed superior stability over a two-year storage period.

TABLE 14
SEC-HPLC and SCX-HPLC Results for FB825 Formulations for Two Years

| | | SEC-HPLC analysis (Main Peak %) | | | | | | | | SCX-HPLC analysis (Main Peak %) | | | | | | | |
|---|---|---|---|---|---|---|---|---|---|---|---|---|---|---|---|---|---|
| Time | Form. | | T = 1 | T = 3 | T = 6 | T = 9 | T = 12 | T = 18 | T = 24 | | T = 1 | T = 3 | T = 6 | T = 9 | T = 12 | T = 18 | T = 24 |
| point | Code | T = 0 | M | M | M | M | M | M | M | T = 0 | M | M | M | M | M | M | M |
| −70° C. | C10 | 99.0 | 98.9 | 99.1 | 99.0 | 99.0 | 98.9 | 98.8 | 98.8 | 53.5 | 52.4 | 53.8 | 54.5 | 55.3 | 54.7 | 55.4 | 54.3 |
| | R6.0 | 99.3 | 99.2 | 99.3 | 99.2 | 99.3 | 99.3 | 99.2 | 99.2 | 54.7 | 53.5 | 54.0 | 54.9 | 55.5 | 54.8 | 54.8 | 54.1 |
| | RR6.0 | 99.3 | 99.3 | 99.3 | 99.1 | 99.3 | 99.3 | 99.4 | 99.3 | 53.7 | 53.3 | 53.6 | 54.5 | 54.8 | 54.7 | 55.2 | 54.2 |
| 5° C. | C10 | 99.0 | 99.0 | 99.1 | 98.9 | 99.0 | 99.0 | 98.4 | 97.5 | 53.5 | 53.8 | 53.9 | 54.1 | 54.9 | 54.3 | 54.0 | 53.9 |
| | R6.0 | 99.3 | 99.0 | 98.9 | 98.5 | 98.7 | 98.6 | 97.5 | 97.0 | 54.7 | 53.0 | 53.7 | 53.8 | 54.3 | 53.6 | 52.5 | 51.0 |
| | RR6.0 | 99.3 | 99.1 | 99.0 | 98.7 | 98.8 | 98.6 | 97.5 | 96.9 | 53.7 | 53.6 | 53.6 | 53.6 | 54.4 | 53.4 | 52.6 | 51.6 |
| 25° C. | C10 | 99.0 | 99.0 | 98.8 | 95.3 | — | — | — | — | 53.5 | 54.7 | 50.2 | 45.7 | — | — | — | — |
| | R6.0 | 99.3 | 98.5 | 97.9 | 93.7 | — | — | — | — | 54.7 | 52.4 | 47.2 | 41.3 | — | — | — | — |
| | RR6.0 | 99.3 | 98.5 | 98.0 | 93.9 | — | — | — | — | 53.7 | 52.8 | 47.6 | 41.2 | — | — | — | — |
| 40° C. | C10 | 99.0 | 97.8 | 93.4 | — | — | — | — | — | 53.5 | 47.7 | 24.3 | — | — | — | — | — |
| | R6.0 | 99.3 | 95.0 | 90.2 | — | — | — | — | — | 54.7 | 37.8 | 17.3 | — | — | — | — | — |
| | RR6.0 | 99.3 | 95.1 | 90.9 | — | — | — | — | — | 53.7 | 37.6 | 19.5 | — | — | — | — | — |

5

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An antibody formulation, comprising:
(a) an antibody binding to a CεmX domain of a membrane-bound IgE at a concentration of about 120-200 mg/ml,
(b) histidine at a concentration of about 5-15 mM,
(c) an amino acid at a concentration of about 1-3.0% (w/v), wherein the amino acid is arginine or threonine;
(d) sodium chloride at a concentration of about 50-100 mM; and
(e) polysorbate 80 at a concentration of about 0.005-0.02%;
wherein the antibody formulation has a pH of about 5.5 to 6.5.

2. The antibody formulation of claim 1, wherein the formulation comprises about 150 mg/ml of the antibody, about 10 mM of the histidine, about 1.25% of the amino acid, about 75 mM of the sodium chloride, and about 0.01% of the polysorbate 80.

3. The antibody formulation of claim 1, wherein the amino acid is arginine and the antibody formulation has a pH of about 6.0 to 6.5.

4. The antibody formulation of claim 1, wherein the amino acid is threonine and the antibody formulation has a pH of about 6.0.

5. The antibody formulation of claim 1, wherein the antibody formulation consists essentially of (a)-(e).

6. The antibody formulation of claim 1, wherein the antibody formulation is free of a sugar- or sugar alcohol-based stabilizer.

7. The antibody formulation of claim 6, wherein the sugar- or sugar alcohol-based stabilizer is sucrose, sorbitol, or trehalose.

8. The antibody formulation of claim 1, wherein the antibody that binds the CεmX domain of a membrane-bound IgE comprises the same heavy chain complementary determining regions (CDRs) as antibody FB825; and/or the same light chain complementary determining regions (CDRs) as antibody FB825.

9. The antibody formulation of claim 8, wherein the antibody is a human antibody or a humanized antibody.

10. The antibody formulation of claim 8, wherein the antibody is a full-length antibody.

11. The antibody formulation of claim 8, wherein the antibody comprises a heavy chain variable region (VH) having the amino acid sequence of SEQ ID NO:2, SEQ ID NO:8, or SEQ ID NO:9, and a light chain variable region (VL having the amino acid sequence of SEQ ID NO:3 or SEQ ID NO:10.

12. The antibody formulation of claim 11, wherein the antibody comprises a VH of SEQ ID NO:9 and a VL of SEQ ID NO:10.

13. The antibody formulation of claim 8, wherein the antibody is an IgG1 molecule.

14. The antibody formulation of claim 13, wherein the antibody comprises a heavy chain comprising the amino acid sequence of SEQ ID NO: 4 or SEQ ID NO:11 and a light chain comprising the amino acid sequence of SEQ ID NO:5 or SEQ ID NO:12.

15. A pre-filled syringe, comprising an antibody formulation set forth in claim 1.

16. The pre-filled syringe of claim 15, wherein the volume of the antibody formulation in the pre-filled syringe is about 1-2 ml.

17. A method for treating a disorder associated with immunoglobulin E (IgE), the method comprising administering to a subject in need thereof an effective amount of the antibody formulation of claim 1.

18. The method of claim 17, wherein the subject receives one dose of the antibody formulation.

19. The method of claim 17, wherein the subject receives at least two doses of the antibody formulation, wherein two consecutive doses are at least 3 months apart.

20. The method of claim 17, wherein the antibody formulation is administered subcutaneously.

21. The method of claim 17, wherein the subject is a human patient having or suspected of having allergic asthma, allergic rhinitis, atopic dermatitis, or hyper IgE syndrome.

22. The method of claim 21, wherein the human patient has atopic dermatitis and optionally wherein the human patient has a low serum IgG4 level.
